(19) United States
(12) Patent Application Publication
(10) Pub. No.: US 2010/0010281 A1
(43) Pub. Date: Jan. 14, 2010

(54) Method and System for Separation and Purification of High-Purity 2,6-Dimethylnaphthalene by Continuous Crystallization

(76) Inventors: Yang Ho Chen, Gyeonggi-do (KR); Young Gyo Choi, Gyeonggi-do (KR)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 12/439,524

(22) PCT Filed: Dec. 20, 2007
(86) PCT No.: PCT/KR2007/006690

§ 371 Date: Apr. 30, 2009

(30) Foreign Application Priority Data
Dec. 29, 2006 (KR) 10-2006-0138696

(51) Int. Cl. C07C 7/14; B01D 9/00

(52) U.S. Cl. 585/804; 422/254

(57) ABSTRACT

Provided is a method for the separation and purification of high-purity 2,6-dimethylnaphthalene from a reaction mixture of dimethylnaphthalenes by continuous crystallization. According to the method, shell-tubetype crystallization apparatuses are used to perform crystallization operations under a continuous flow of a reaction mixture of dimethylnaphthalenes, which is obtained from the synthesis of dimethylnaphthalenes using o-xylene and butadiene as starting materials. As a result, high-purity 2,6-dimethylnaphthalene is separated and purified in a high yield from the reaction mixture. In addition, the method is advantageous in terms of energy saving when compared to conventional separation methods and enables continuous separation and purification of 2,6-dimethylnaphthalene on an industrial scale. A system for implementing the method is further provided.

### TECHNICAL FIELD

The present invention relates to a method for separating and purifying high-purity 2,6-dimethylnaphthalene from a reaction mixture of dimethylnaphthalenes by continuous crystallization. More specifically, the present invention relates to a method and a system for separating and purifying high-purity 2,6-dimethylnaphthalene in a high yield from a reaction mixture of dimethylnaphthalenes, which is obtained from the synthesis of dimethylnaphthalenes using o-xylene and butadiene as starting materials, that uses shell-tubetype crystallization apparatuses to perform crystallization operations under a continuous flow of the reaction mixture.

### BACKGROUND ART

2,6-Naphthalenedicarboxylic acid (2,6-NDA) is a monomer of highly functional polyethylene naphthalate (PEN) resins and is well known as a raw material for liquid crystal polymers. PEN resins are said to offer excellent physical properties in terms of heat resistance, tensile strength and gas barrier properties over polyethylene terephthalate (PET) resins that are currently used in a wide variety of applications.

It is known that 2,6-naphthalenedicarboxylic acid is produced from various raw materials, for example, dimethylnaphthalenes (DMN), diethylnaphthalenes, dipropylnaphthalenes and dibutylnaphthalenes. The alkylnaphthalenes other than dimethylnaphthalenes are rarely used to produce 2,6-naphthalenedicarboxylic acid from the viewpoint of economic efficiency because of their low reactivity and selectivity in the oxidation for the production of 2,6-naphthalenedicarboxylic acid.

The oxidation of 2,6-dimethylnaphthalene is known to be the most effective method for the production of 2,6-naphthalenedicarboxylic acid using dimethylnaphthalenes and is considered to be the most effective synthetic route. Thus, there is a continuous need to develop more efficient methods for the separation and purification of 2,6-dimethylnaphthalene.

On the other hand, when it is intended to produce 2,6-naphthalenedicarboxylic acid by the oxidation of 2,6-dimethylnaphthalene as a raw material, the quality of the final product is greatly affected by the purity of the raw material. The presence of slight amounts of impurities in 2,6-dimethylnaphthalene leads to a deterioration in the physical properties (e.g., purity, color, etc.,) of 2,6-naphthalenedicarboxylic acid. Accordingly, a high purity (=99%) of 2,6-dimethylnaphthalene is required for the production of 2,6-naphthalenedicarboxylic acid with good physical properties. To this end, it is necessary to separate and purify 2,6-dimethylnaphthalene from a reaction mixture of dimethylnaphthalenes.

Many methods are widely employed at present to separate and purify dimethylnaphthalene isomers and examples thereof include separation by complexation, adsorptive separation and fractional recrystallization. The fractional recrystallization is a method in which 2,6-dimethylnaphthalene is separated through crystallization-recrystallization using a suitable solvent at relatively low cost.

Dimethylnaphthalenes are well known to form eutectic mixtures. For example, a binary eutectic mixture of 2,6-dimethylnaphthalene and 2,7-dimethylnaphthalene is formed in a molar ratio of 41.5:58.5 and a binary eutectic mixture of 2,6-dimethylnaphthalene and 2,3-dimethylnaphthalene is formed in a molar ratio of 47.5:52.5. Since the amount of 2,6-dimethylnaphthalene produced is theoretically determined from the composition of dimethylnaphthalenes, sufficient high purity and yield of 2,6-dimethylnaphthalene cannot be achieved by typical separation methods, such as recrystallization. Further, the separation of 2,6-dimethylnaphthalene is troublesome and time-consuming, and the final purity of 2,6-dimethylnaphthalene is relatively low. For these problems, there have been few reviews on recrystallization as a practical separation process.

Dimethylnaphthalene isomers have very similar boiling points (approx. 262.0° C.), and thus it is very difficult to separate 2,6-dimethylnaphthalene from other dimethylnaphthalene isomers by common distillation techniques. The separation of 2,6-dimethylnaphthalene is known to inevitably involve technical difficulties in achieving high purity, low recovery and considerable separation and purification costs.

EP 0 336 564 A1 (1989) discloses a process for separating 2,6-dimethylnaphthalene comprising the three steps of preliminary treatment of a naphthalenic mixture as a starting material, distillation and crystallization under pressure. However, 2,6-dimethylnaphthalene separated by the process was reported to have a low purity of 98% or less, which does not meet the purity requirement for the production of 2,6-naphthalenedicarboxylic acid.

### DISCLOSURE OF INVENTION

### Technical Problem

The present invention has been made in an effort to solve the problems of the prior art, and it is one object of the present invention to provide a method for continuously separating and purifying high-purity 2,6-dimethylnaphthalene from a reaction mixture of dimethylnaphthalenes in a more economical and efficient manner that uses shell-tube type crystallization apparatuses to crystallize and recrystallize the reaction mixture.

It is another object of the present invention to provide a shell-tube type crystallization apparatus used in the separation and purification method of 2,6-dimethylnaphthalene.

### Technical Solution

In accordance with one aspect of the present invention for accomplishing the above objects, there is provided a method for continuously separating and purifying 2,6-dimethylnaphthalene from a reaction mixture of dimethylnaphthalenes, the method comprising crystallizing and recrystallizing the reaction mixture using shell-tube type crystallization apparatuses.

In an embodiment of the present invention, the crystallization and recrystallization may be performed in two or multiple crystallization stages.

In accordance with another aspect of the present invention, there is provided a shell-tube type crystallization apparatus used in the method.

### ADVANTAGEOUS EFFECTS

The method of the present invention uses shell-tube type crystallization apparatuses to separate and purify 2,6-dimethylnaphthalene (2,6-DMN). Heat of fusion, which corresponds to about one-fifth of heat of vaporization used for distillation, is utilized in the method of the present invention to save energy consumption. In addition, according to the method of the present invention, simple solid-liquid separation operations are employed to separate high-purity 2,6-DMN in a high yield. Furthermore, the method of the present invention is implemented using a simple separation and purification system in a simple manner, leading to reduced fixed investment and production costs. Therefore, the method of the present invention is economically advantageous. Moreover, solution crystallization can be additionally performed to effectively separate high-purity 2,6-DMN.

### BRIEF DESCRIPTION OF THE DRAWINGS

FIG. 1 is a process flow diagram schematically illustrating a method for the separation and purification of 2,6-dimethylnaphthalene by continuous crystallization according to an embodiment of the present invention.

FIG. 2 is a see-through perspective view of a shell-tube type crystallization apparatus according to the present invention and illustrates the flows of a raw material and a coolant in the crystallization apparatus.

FIG. 3 shows a see-through perspective view (3a) of a shell-tube type crystallization apparatus according to the present invention and cross-sectional views (3b, 3c) of internal scrapers of the crystallization apparatus.

### BRIEF EXPLANATION OF ESSENTIAL PARTS OF THE DRAWINGS

1: DMN mixture reservoir, 2, 9: Solvent mixing tanks
3: Solvent reservoir, 4, 11: Crystallization apparatuses
5, 12: Centrifugal separators, 6. 13: Screw conveyers
7: Crystal melting tank, 8: Stock solution reservoir
14, 15: Solvent reservoirs, 16, 17: Refrigerator

### BEST MODE FOR CARRYING OUT THE INVENTION

Exemplary embodiments of the present invention will now be described in greater detail with reference to the accompanying drawings.

In one aspect, the present invention provides a method for separating and purifying high-purity 2,6-dimethylnaphthalene in a high yield from a reaction mixture of dimethylnaphthalenes, which is obtained from the synthesis of dimethylnaphthalenes using o-xylene and butadiene as starting materials, that uses shell-tube type crystallization apparatuses to perform crystallization operations under a continuous flow of the reaction mixture.

Particularly, the content of 2,7-dimethylnaphthalene in the reaction mixture of dimethylnaphthalenes is as low as 0.2%, resulting in an improvement in the yield and purity of 2,6-dimethylnaphthalene, and ethanol is used as a solvent to achieve improved separation efficiency of 2,6-dimethylnaphthalene.

The isomeric mixture of dimethylnaphthalenes as a raw material used in the method of the present invention includes ten different isomers, such as 2,6-DMN, 1,6-DMN and 1,5-DMN, obtained from the isomerization of the dimethylnaphthalenes, high boiling point hydrocarbons and low boiling point hydrocarbons. The composition and physical properties of the constituent compounds are indicated in Table 1.

TABLE 1
Composition and physical properties of dimethylnaphthalene
isomers and other compounds

| | Content | Boiling point | Melting point |
|---|---|---|---|
| Compound | (wt %) | (° C.) | (° C.) |
| 2,6-DMN | 40.0-50.0 | 262 | 112 |
| 1,5-DMN | 20.0-25.0 | 269 | 82 |
| 1,6-DMN | 20.0-25.0 | 266 | −16 |
| 2,7-DMN | 0.00-0.10 | 262 | 98 |
| 2,3-DMN | 0.00-0.10 | 269 | 104 |
| 1,7-DMN | 0.00-0.10 | 263 | −14 |
| 1,8-DMN | 0.00-0.10 | 270 | 65 |
| 1,2-DMN | 0.00-0.10 | 271 | −3.5 |
| 1,4-DMN | 0.00-0.10 | 265 | 6 |
| 1,3-DMN | 0.00-0.10 | 265 | −4.2 |
| Low boiling point | 9.1-10.0 | — | — |
| hydrocarbons | | | |
| High boiling point | 9.2-10.0 | — | — |
| hydrocarbons | | | |

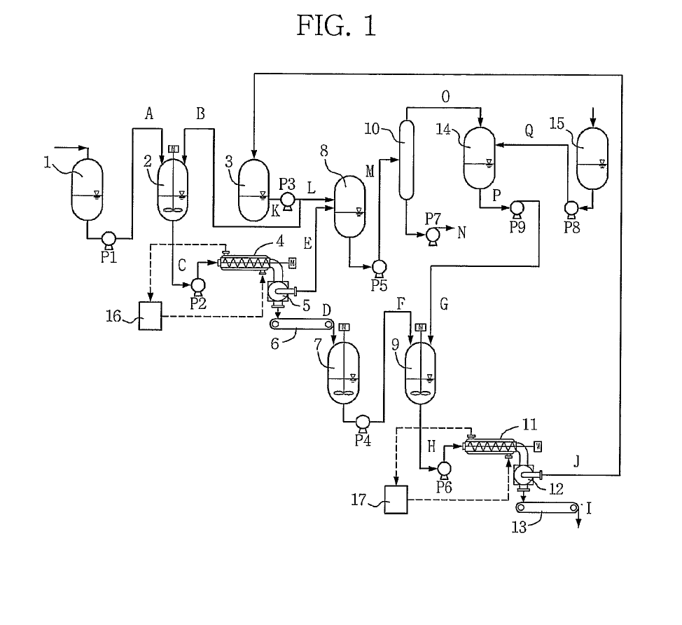

FIG. 1 is a process flow diagram schematically illustrating a method for the separation and purification of 2,6-dimethylnaphthalene by continuous crystallization according to an embodiment of the present invention.

Referring to FIG. 1, an isomeric mixture as a starting raw material rich in 2,6-DMN undergoes isomerization and is sent to a DMN mixture reservoir 1 (Stage A). The DMN isomeric mixture is transferred to a solvent mixing tank 2 by means of a pump P1. The DMN isomeric mixture is mixed with a solvent in the solvent mixing tank 2. A C₁-C₈ alcohol can be used as the solvent. The use of ethanol is preferred.

As a solvent for primary crystallization, a stock solution separated in subsequent secondary crystallization is used. The stock solution is transferred to a stock solution reservoir 3 and is then transferred to the solvent mixing tank by means of a pump P3 (Stage B). The solvent mixing tank is maintained at a temperature of 60° C. in order to maintain the raw material in a molten state. A pump P2 is operated to introduce the raw material mixture into a first crystallization apparatus 4 composed of shell-tube crystallizers (Stage C).

The primary crystallization is accomplished while the raw material mixture is continuously supplied to the first crystallization apparatus 4 by means of the pump P2 and a coolant from a refrigerator 16 is circulated through the shell-tube of the first crystallization apparatus. Crystals and a stock solution obtained after the primary crystallization are introduced into a centrifugal separator 5 directly connected to the first crystallization apparatus. The stock solution separated by the centrifugal separator is transferred to a first stock solution reservoir 8 and the separated crystals are conveyed to a first crystal melting tank 7 by means of a screw conveyer 6 (Stage D).

The crystals are in a molten state in the first crystal melting tank at 80 C and are transferred to a second solvent mixing tank 9 by means of a pump P4. A solvent used for secondary crystallization flows from a pure solvent intermediate reservoir 14 to the second solvent mixing tank 9 along a line P and G by means of a pump P9. The second solvent mixing tank is maintained at a temperature of 60° C. The dissolved raw material is transferred from the second solvent mixing tank to a second crystallization apparatus 11 along a line H by means of a pump P6. The second crystallization apparatus has the same structure as the first crystallization apparatus, except that the overall capacity and the size of the crystallization apparatuses are varied according to the solvent ratios. The second crystallization apparatus is cooled by a refrigerator 17.

A solution containing 2,6-DMN crystals obtained after the secondary crystallization is separated into a stock solution and 2,6-DMN crystals by a centrifugal separator 12. The separated 2,6-DMN crystals are conveyed by a screw conveyer 13. The separated stock solution is transferred to the second stock solution reservoir 3 along a line J. A portion of the stock solution introduced into the stock solution reservoir is used for the primary crystallization and the remaining portion thereof is transferred to the first stock solution reservoir 8. The stock solution transferred to the first stock solution reservoir is crystallized from the solvent and is transferred to a solvent separation column via a pump P5 to separate the remaining DMN isomeric mixture (Stage M).

The DMN isomeric mixture drawn from the bottom of the solvent separation column flows along a line N by means of a pump P7. The solvent drawn from the top of the solvent separation column is sent to the second solvent reservoir 14 along a line O. A new solvent is fed into a first solvent reservoir 15 and is then transferred to the second solvent reservoir along a line Q to compensate for a loss of the solvent through the overall procedure.

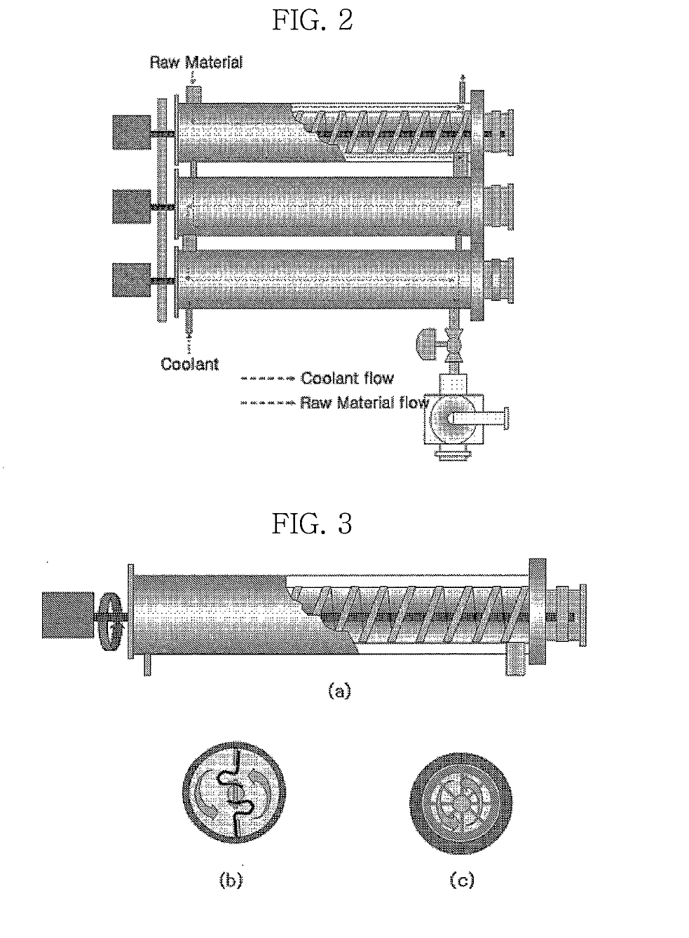

FIG. 2 is a see-through perspective view of one of the shell-tube type crystallization apparatuses and illustrates the flows of the raw material and the coolant in the crystallization apparatus. The coolant is circulated in a direction opposite to the flow of the raw material to cool the crystallization apparatus and to form 2,6-DMN crystals. The temperatures of the raw material at inlet and outlet ports of the first crystallization apparatus are adjusted to 50 to 60° C. and −10 to 0° C., respectively. The temperatures of the coolant at inlet and outlet ports of the first crystallization apparatus are adjusted to −15 to −10° C. and 30 to 40° C., respectively. The temperature conditions of the second crystallization apparatus are the same as those of the first crystallization apparatus.

The reason why the temperatures of the raw material at the inlet and outlet ports of the crystallization apparatus are limited to the respective ranges defined above is because the crystallization is performed under the temperature conditions to achieve optimal purity and yield of the crystals depending on the solubility of the DMN isomeric mixture in the solvent according to the composition of the DMN isomeric mixture. When the temperature of the raw material at the inlet port of the crystallization apparatus is higher than 60° C., the vaporization of the solvent occurs, which causes a difficulty in the transfer of the raw material, and the cooling temperature must be further lowered, which requires a large quantity of energy for the crystallization. When the temperature of the raw material at the inlet port of the crystallization apparatus is lower than 50° C., the raw material is not sufficiently dissolved, which makes it impossible to normally perform the crystallization and causes a reduction in the purity of the crystals. On the other hand, when the temperature of the raw material at the outlet port of the crystallization apparatus is higher than 0° C., the purity of the crystals is increased but the yield thereof is lowered. When the temperature of the raw material at the outlet port of the crystallization apparatus is lower than −10° C., the desired purity of the crystals cannot be attained.

The reason why the temperatures of the coolant at the inlet and outlet ports of the crystallization apparatus are limited to the respective ranges defined above is the same as the reason why the temperatures of the raw material are limited. That is, sufficient yield and purity are attained when the crystallization is performed at the above temperature ranges of the coolant.

However, the ratio of the solvent to the mixture contained in the stock solution used in the primary crystallization is adjusted to 3-4:1. As for the secondary crystallization, ethanol as the solvent and the mixture are mixed together in a weight ratio of 5-8:1 in the mixing tank, and then the mixture is introduced into the second crystallization apparatus. The solvent ratios and the temperature conditions of the respective crystallization apparatuses are appropriately controlled depending on the desired purity and yield of the crystals.

The reason why the crystallization is performed in the given solvent ratio range is to achieve desired purity and yield in the respective crystallization stages according to the variation in the composition of the raw material introduced into the corresponding crystallization apparatus.

FIG. 3 shows a see-through perspective view (3a) of the shell-tube type crystallization apparatus used in the method of the present invention and cross-sectional views (3b, 3c) of internal scrapers of the crystallization apparatus. The reason for the use of the shell-tube type crystallization apparatus is to continuously perform the crystallization. The internal scrapers are designed to prevent 2,6-DMN crystals from aggregation in a plate form and being adhered to the cooled inner wall surfaces. The internal scrapers serve to sufficiently enhance the cooling efficiency of the raw material and are means for the continuous transfer of the raw material.

The purity and the yield of the produced 2,6-DMN are calculated by Equations 1 and 2:

$$\begin{matrix}Purity ( \% ) = \frac{Weight of 2 , 6 - DMN in the mixture}{Total weight of the mixture} \times 100 & ( 1 ) \\ Yield ( \% ) = \frac{\begin{matrix}Weight of 2 , 6 - \\ DMN obtained by crystallization\end{matrix}}{\begin{matrix}Weight of 2 , 6 - \\ DMN contained in the \\ sample before crystallization\end{matrix}} \times 100 & ( 2 )\end{matrix}$$

The purity of the DMN isomeric mixture used in the present invention and the purities of the materials separated and purified in the respective stages are analyzed by gas chromatography.

In another aspect, the present invention is directed to a shell-tubetype crystallization apparatus used in the method.

The shell-tube type crystallization apparatus comprises double-pipe type crystallizers made of stainless steel, each of which is composed of an internal processing tube (tube-side) through which the raw material flows and an external jacket (shell-side) through which the coolant flows, pedals in the form of a spring (FIG. 3b) or screws (FIG. 3c) as internal scrapers, and motors for rotating the screws or pedals.

The shell-tube type crystallizers may be made of various materials. The double-pipe type crystallizers in the form of a pipe can be easily manufactured and enable continuous crystallization. Since the shell-tubecrystallization apparatus has a temperature gradient between the inlet and outlet ports, the degree of supersaturation during the crystallization is appropriately varied, which has a positive influence on the shape of the crystals and the purity of the crystals during the separation.

The coolant flows in the external jacket (shell) in a direction opposite to the flow of the raw material to property control the heat transfer efficiency and the degree of supersaturation during the crystallization. Draft tubes or baffles are installed in general cooling crystallizers to increase the heat transfer area of the crystallizers. However, the use of draft tubes or baffles has limitations in large-capacity crystallization apparatuses on an industrial scale. In contrast, the crystallization apparatus of the present invention comprises small-size crystallizers to achieve increased heat transfer area, which is a significant feature inshell-tubecrystallization apparatuses, despite considerable installation costs. In addition, the crystallization apparatus of the present invention has the advantages of extended double-pipe jacketed crystallizers, improved heat transfer efficiency of the coolant, and high heat transfer efficiency per unit area.

The internal transfer screws or pedals attached to the respective motors are readily constructed for low-speed operation and need a relatively small quantity of energy when compared to general crystallizers equipped with agitators. Moreover, the screws or pedals effectively prevent 2,6-DMN crystals from being adhered to the wall surfaces of the crystallizers, and as a result, no drop in heat transfer area is exhibited.

The internal screw shown in FIG. 3 is configured such that the distance between the internal screw and the inner wall of the crystallizer is below 1 cm depending on the size of the crystallization apparatus to optimally transfer the crystals and the stock solution from the inlet port to the outlet port. In addition, the screw serves to remove 2,6-DMN crystals adhered to the inner wall surfaces of the crystallizer to make the heat transfer efficiency better.

The ends of the screw are enhanced using a polymeric plastic (e.g., Teflon) to prevent the inner wall of the crystallizer from being damaged. The internal scraper in a pedal form performs the same role as the screw and has a spring structure to prevent the inner wall of the crystallizer from being damaged.

The crystallization apparatus of the present invention is configured to allow the raw material and the coolant to flow in multiple stages, as shown in FIG. 2, taking into consideration the volume of the crystallizers, heat transfer efficiency and crystal growth rate, etc. in order to control the retention time and crystal growth rate. Therefore, the crystallization apparatus of the present invention can be used to continuously separate and purify high-purity 2,6-DMN in a sufficient large amount within a small space.

In conclusion, according to the method and the system of the present invention, heat of fusion, which corresponds to about one-fifth of heat of vaporization used for distillation, is utilized to save energy consumption. In addition, according to the present invention, simple solid-liquid separation operations are employed to separate high-purity 2,6-DMN in a high yield.

### MODE FOR THE INVENTION

Hereinafter, the present invention will be explained in more detail with reference to the following examples. However, these examples are given for the purpose of illustration only and are not intended to restrict or limit the scope of the present invention.

### EXAMPLES

### Example 1

A reaction mixture of dimethylnaphthalenes containing an average of 42.53% by weight of 2,6-DMN was transferred at a rate of 15 kg/hr to the first solvent mixing tank. The reaction mixture was mixed with the stock solution separated in secondary crystallization, which was used for primary crystallization, until the average solvent ratio reached 4:1. The raw material mixture was introduced into and crystallized in the first crystallization apparatus. At this time, the temperature of the outlet port of the first crystallization apparatus was adjusted to 0° C. After the primary crystallization, centrifugation was performed to obtain crystals. The crystals were sampled and analyzed. The analytical results are shown in Table 2. The crystals were dissolved in the melting tank at 80° C. and transferred to the second solvent mixing tank. Ethanol was used to dissolve the crystals in a ratio of 8:1 in the second solvent mixing tank, and then the solution was transferred at a flow rate of 60 kg/hr to the second crystallization apparatus. At this time, the temperature of the outlet port of the second crystallization apparatus was adjusted to 0° C. After the secondary crystallization, centrifugation was performed to obtain crystals with a purity of 99.20% by weight in a yield of 95.6%.

TABLE 2

| | DMN isomeric | Primary crystal- | Secondary crystal- |
|---|---|---|---|
| | mixture | lization | lization |
| Purity (wt %) | 42.53 | 75.91 | 99.20 |
| Yield (%) | 100.0 | 94.9 | 95.6 |

### Example 2

To evaluate the influence of the structure of internal scrapers on crystallization, crystals were obtained in the same manner as in Example 1, except that scrapers having different structures were used. The average values of the obtained results are shown in Table 3.

TABLE 3

| | DMN isomeric | Use of internal | Use of internal |
|---|---|---|---|
| | mixture | scraper (FIG. 3b) | scraper (FIG. 3c) |
| Purity (wt %) | 42.78 | 99.21 | 99.45 |
| Yield (%) | 100.0 | 85.9 | 88.9 |

### Comparative Example 1

In this example, general batch jacketed cooling crystallization apparatuses were used instead of the shell-tubetype crystallization apparatuses. The first crystallization apparatuses were jacketed crystallization apparatuses equipped with baffles, and the second crystallization apparatuses were crystallization apparatuses equipped with draft tubes to increase the cooling efficiency and heat transfer area.

45 kg of a DMN reaction mixture of dimethylnaphthalenes containing an average of 42.05% by weight of 2,6-DMN was transferred to the first crystallization apparatus. The reaction mixture was mixed with the stock solution separated in secondary crystallization, which was used for primary crystallization, until the average solvent ratio reached 4:1. After completion of the transfer, stirring was conducted for 10 minutes to dissolve the mixture. The amount of the mixture transferred for primary crystallization was 225 kg. The raw material mixture was introduced into and crystallized in the first crystallization apparatus. At this time, the crystallization temperature was 0° C. The crystallization was conducted in a batch crystallization mode for three hours. After the primary crystallization, centrifugation was performed to obtain primary crystals. Ethanol was used to dissolve the primary crystals in a ratio of 8:1 in the second solvent mixing tank, and then the solution was transferred to the second crystallization apparatus. At this time, the crystallization temperature was adjusted to 0° C. After the secondary crystallization, centrifugation was performed to obtain secondary crystals. The obtained results are shown in Table 4.

TABLE 4

| | | | Crystallization |
|---|---|---|---|
| | | Jacketed crystallization | apparatuses |
| | DMN isomeric | apparatuses equipped | equipped with |
| | mixture | with baffles | draft tubes |
| Purity (wt %) | 42.05 | 98.21 | 95.38 |
| Yield (%) | 100.0 | 45.1 | 68.9 |

Although the present invention has been described herein with reference to the foregoing preferred embodiments, those skilled in the art will appreciate that various modifications and changes are possible without departing from the spirit of the present invention as disclosed in the accompanying claims. It is to be understood that such modifications and changes are within the scope of the present invention.

1. A method for separating and purifying 2,6-dimethylnaphthalene from a reaction mixture of dimethylnaphthalenes as a raw material the method comprising crystallizing and recrystallizing the reaction mixture using shell-tube type crystallization apparatuses.

2. The method according to claim 1, wherein the crystallization and the recrystallization are performed in two or more crystallization stages.

3. The method according to claim 1, wherein the flow of a coolant in the crystallization apparatus is opposite to that of the raw material.

4. The method according to claim 1, wherein the shell-tube type crystallization apparatuses are configured to remove the crystals adhered to the inner wall surfaces and to continuously transfer the raw material and the crystals during the crystallization and recrystallization.

5. The method according to claim 1, wherein the temperatures of the raw material at inlet and outlet ports of each of the crystallization apparatuses are adjusted to 50 to 60° C. and −10 to 0° C. respectively, during the crystallization and recrystallization.

6. The method according to claim 1 wherein the temperatures of a coolant at inlet and outlet ports of each of the crystallization apparatuses are adjusted to −15 to −10° C. and 30 to 40° C., respectively, during the crystallization and recrystallization.

7. The method according to claim 2, wherein a solvent for primary crystallization is mixed with a stock solution used in secondary crystallization to obtain crystals.

8. The method according to claim 2, wherein the ratio of the reaction mixture of dimethylnaphthalenes to a solvent for primary crystallization is in the range of 1:3 to 1:4 and the ratio of the reaction mixture to the solvent for secondary crystallization in the range of 1:5 to 1:8.

9. The method according to claim 1, wherein the purity of the crystals obtained after primary crystallization is 70-80% by weight and that of the crystals obtained after secondary crystallization is higher than 99% by weight.

10. A shell-tubetype crystallization apparatus, comprising double-pipe type jacketed crystallizers and surface scraper internal screws or spring pedals equipped with motors.
